(19) United States
(12) Patent Application Publication
(10) Pub. No.: US 2015/0258275 A1
(43) Pub. Date: Sep. 17, 2015

(54) INTRAVENOUS MEDICATION DELIVERY SYSTEM

(71) Applicant: Susan J. MACK, Washington, DC (US)

(72) Inventor: Andreas Kotzur, Washington, DC (US)

(21) Appl. No.: 14/435,051

(22) PCT Filed: Mar. 13, 2014
(86) PCT No.: PCT/US14/25557

§ 371 Date: Apr. 10, 2015

Related U.S. Application Data
(60) Provisional application No. 61/792,936, filed on Mar. 15, 2013.

(51) Int. Cl. A61M 5/172; A61M 5/168

(52) U.S. Cl. CPC A61M 5/172; A61M 5/1723; A61M 5/16813; A61M 2205/50; A61M 2230/005; A61M 2230/06; A61M 2230/43; A61M 2230/30; A61M 2205/3576

(57) ABSTRACT

The present invention relates generally to delivery devices including medication containers, medication trays, valve trays, intravenous delivery trays, a medication delivery unit for delivering a medication, and methods of making and using such delivery devices.

### RELATED APPLICATION

This application claims priority from U.S. Provisional Patent Application No. 61/792,936, filed on Mar. 15, 2013, in the United States Patent and Trademark Office, the disclosure of which is hereby incorporated herein in its entirety by reference.

### FIELD OF THE INVENTION

The present invention relates generally to delivery devices including medication containers, medication trays, valve trays, intravenous trays, a medication delivery unit for delivering a medication, and methods of making and using such delivery devices.

### BACKGROUND OF THE INVENTION

Medical treatments often involve solutions or suspensions of drugs or other medication to be injected into the human body. Mixing and injecting such solutions can be extremely expensive and inaccurate. Thus, there are a number of problems with current methods of intravenous medication delivery.

During hospital stays and operations, patients frequently receive intravenous fluids and medications. These medications are drawn up and administered by trained personal, such as nurses and physicians. However, there is a high incidence of medication errors due to a number of factors; all of which are likely to be a result of operator error.

Traditionally, intravenous and parenteral medications are filled into vials and containers of various shapes, sizes and consistencies by pharmaceutical companies. The operator then draws up the medication from the container into a syringe and then administers the drug either into an IV or an epidural line. Some of these containers are multi dose and some are single dose, meaning that they have to be discarded after a single use. During the transfer of drug from the container to the syringe there is an opportunity for bacterial contamination. Also, there is room for operator error, to either administer the wrong drug, or to miscalculate the dose of drug given, or to mistakenly administer a different amount from the one intended.

Since the traditional transfer of drug from a vial to the patient involves needles, there is also the potential of a needle stick injury to the healthcare worker.

The present invention is directed to overcoming these and other deficiencies in the art.

### SUMMARY OF THE INVENTION

The present invention will improve the accuracy and sterility of medication delivery. It will also significantly reduce operator errors. Accurate delivery of a specified dose of intravenous or parenteral medication can be achieved by the delivery systems of the present invention.

In a first aspect the present invention is related to a medication tray. The medication tray can include a chassis adapted to hold at least one medication container wherein the medication container comprises a medication. The medication tray can further include a means capable of delivering the medication from the medication container to intravenous (IV) tubing. The tray can also include a communication interface capable of transmitting and receiving information. The communication interface can be adapted such that it is in communication with the medication container and a controller. The controller is configured to process the information regarding the medication to be delivered and control delivery of the medication from the medication container to the intravenous tubing.

The medication containers according to the present invention can also include at least one connector. The connector can be configured such that the controller is in communication with the connector. The connector on the medical container can be configured such that it is communication with the controller via the communication interface on the medication tray. The connector can be in the form of an electrical connector such that the connector is capable of exchanging information with the controller.

In a second aspect the present invention is related to an intravenous tray. The tray can be adapted such that it can detachably couple to the chassis of the medication tray or the valve tray of the present invention. The intravenous tray includes a first input port for a fluid to come in and a second output port for the fluid to go out. The fluid can be a carrier fluid used for IV administration. The tray also includes a plurality of delivery ports arranged on the tray such that each delivery port is capable of fluidic contact with the first input port and the second output port. The delivery ports may be designed such that they are capable of detachably coupling to a plurality of means capable of delivering the medication. The delivery ports can also be designed such that they are fixed to the means capable of delivering the medication.

In a third aspect the present invention is related to a valve which is capable of coupling to the means capable of delivering the medication from the medication container. The valve is capable of detachably coupling to said means or it can be fixed to said means. The valve can be designed such that it allows unidirectional flow of medication from the medication container to the intravenous tubing or intravenous tray. The valve can also be designed such that it includes a means for sterilizing the means capable of delivering the medication. The valves can be designed such that they have a first input port for fluidic contact with the means capable of delivering the medication from the medication container. The valves can also be designed such that they themselves have a means capable of delivering the medication. The valves can also be designed such that they have a second output port.

In a fourth aspect the present invention is related to a valve tray which includes a plurality of detachable valves arranged on the valve tray which includes a plurality of detachable valves. The valves are arranged on a valve tray such that the first input ports of a plurality of detachable valves are capable of coupling with a plurality of medication containers. The coupling can be achieved via container access ports of the medication containers.

In a fifth aspect the present invention is related to a medication delivery unit which includes a housing adapted to hold at least one medication tray. The delivery unit can also include at least one module capable of coupling with the communication interface and the controller such that the controller is capable of transmitting and receiving information regarding a medication to be delivered and control delivery of the medication from the medication container to the intravenous tubing. The medication delivery unit can also be automated using programmed computers and other such systems which are capable of controlling the medication delivery unit.

In a sixth aspect the present invention is related to a method of delivering a medication to a patient including loading the medication into the medication tray of the medication delivery unit of the present invention. The method also includes transmitting information regarding the medication to the controller.

In a seventh aspect the present invention is related to a method of delivering a medication to a patient including loading the medication into the medication tray of the present invention. The method also includes transmitting information regarding the medication to the controller.

These and other aspects and embodiments of the present invention, as well as additional inventive features, will be apparent from the description of the invention provided herein.

### BRIEF DESCRIPTION OF THE DRAWINGS

FIG. 1 shows an exemplary embodiment of the medication tray of the present invention.

FIGS. 2A-B show an exemplary embodiment of the medication containers of the present invention. FIG. 2A shows a medication container, a one way valve, and a delivery tubing. FIG. 2B shows a medication container that is coupled to the delivery tubing via a one way valve.

FIG. 3A shows an exemplary embodiment of a valve tray and FIG. 3B shows an embodiment of the valves of the present invention.

FIG. 4 shows an exemplary embodiment of the intravenous tray of the present invention.

FIG. 5 shows an exemplary embodiment of a housing for medication delivery units of the present invention.

FIG. 6 shows another exemplary embodiment of the medication delivery unit of the present invention.

### DETAILED DESCRIPTION OF THE INVENTION

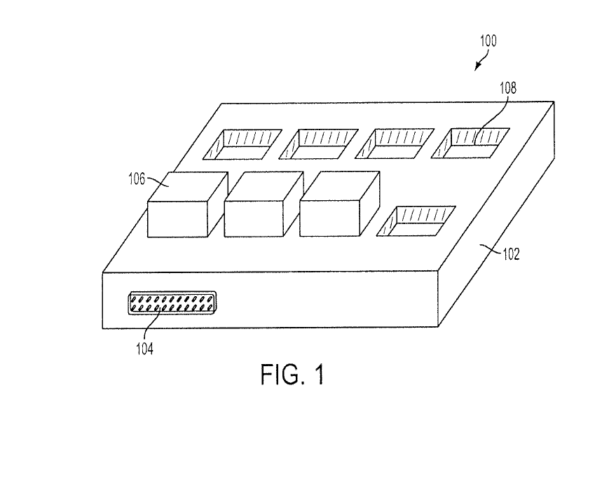

In a first aspect the present invention is related to a medication tray. The medication tray can include a chassis adapted to hold at least one medication container wherein the medication container comprises a medication. The medication tray can further include a means capable of delivering the medication from the medication container to intravenous (IV) tubing. One embodiment of medication tray 100 of the present invention is shown in FIG. 1. Medication tray 100 has a chassis 102 that includes, e.g., eight slots 108 for holding medication containers 106. Slot 108 is adapted to hold a medication tray 106. Three of the eight slots are occupied by medication containers.

The tray can also include a communication interface capable of transmitting and receiving information. In FIG. 1, an example of a communication interface is shown as element 104. The communication interface can be adapted such that it is in communication with the medication container and a controller. The controller is configured to process the information regarding the medication to be delivered and control delivery of the medication from the medication container to the intravenous tubing.

The medication containers according to the present invention can also include at least one connector. The connector can be configured such that the controller is in communication with the connector. The connector on the medical container can be configured such that it is communication with the controller via the communication interface on the medication tray.

The intravenous tubing from the medication tray can be connected to a patient via an intravenous needle or a cannula in a patient. The intravenous tubing can be designed to facilitate removal of air from a patient IV line connectable to a patient and to perform a self-priming function of a drip chamber. As used herein, the term “medication” generally refers to intravenous or parenterally-dispensed solutions.

The medication tray can be designed such that medication container can be ejected from the chassis. The medication containers according the present invention can be designed such that they are reusable or can be discarded after the medication is used.

In one embodiment the medication tray of the present invention may also include a fluidic channel which is connected to each individual medication container. The fluidic channel can be primed with a carrier fluid before a medication container is attached to the medication tray. In one embodiment the fluidic channel is a tubing, for example, an intravenous tubing. The fluidic channel according to the present invention is capable of connecting/coupling to an intravenous tubing. In one embodiment the medication tray includes a tubing which has at least one delivery port capable of coupling with the means capable of delivering the medication.

The carrier fluid according to the present invention can be any pharmaceutically acceptable fluid, for example, a normal saline solution.

The means capable of delivering the medication according to the present invention can be, for example, an opening, a nozzle, a catheter, a plunger with an opening, a valve with an opening, or a container access port which allows flow of medication from the medication container to the intravenous tubing. The container access port can be designed such that it allows medicine to flow at a certain rate into the tubing. The means for delivering the medication can also be, for example, a device such as a container valve which allows unidirectional flow of the medication from the medication container to the intravenous tubing. The valve can be connected to the controller such that the controller can control the medication flow from the valve. This means for delivering the medication can also be designed such that it has mechanisms for turning the flow of medication on and/or off. The mechanism can be placed under the control of the controller. The mechanism can be, for example, an electrical switch which can be turned on and off to control the delivery of the medicine. The mechanism can also be, for example, a stopper or a plunger which can be placed in a position such that it can block the flow of medicine. The stopper can be controllably removed to allow the medicine to flow. The means for delivering the medication can also be, for example, a drip chamber wherein the chamber can be in fluidic contact with the tubing. The means for delivering the medication can also be a roller pump system as well as methods used in, for example, inkjet printers, i.e., piezoelectric methods or thermal methods. The means for delivering the medication can also be a syringe driver system where a medication is expelled by using a nonreciprocating piston driven by a motor.

The medication containers of the present invention can be filled with intravenous and parenteral medications. The medication containers can be standardized such that they are capable of being inserted into the medication tray. The medication tray can in turn be designed such that it is capable of being slotted or inserted into a medication delivery unit.

The medication containers can be prefilled by, for example, a drug company that supplies the drug, or they can be aseptically filled by a trained operator. The medication can also be filled in the container by a pharmacy or a pharmacist, if they have the adequate training or capability. Alternatively, the medication containers can be designed such that they have a docking/delivery system inside them which can be connected or coupled to regular or already available drug vials. For example, a regular vial can be connected to the medication container of the present invention via an adapter where the adapter is capable of connecting to the means for delivering the medication to the tubing.

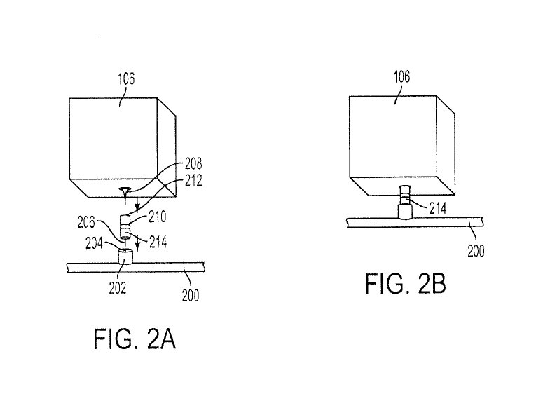

FIG. 2A shows one embodiment of medication container 106 of the present invention. Medication container 106 can couple to medication tray 100 via tubing 200. The tubing has a delivery port 202 which includes an access membrane 204. The tubing 200 is adapted to be placed inside medication tray 100 and to detachably couple with medication container 106 through delivery port 202. Access membrane 204 contains an antiseptic material and is capable of sterilizing means capable of delivering the medication 208 upon coupling.

Medication container 106 also includes a means capable of delivering the medication 208, such as, needle/catheter. Needle 208 is adapted to couple with delivery port 202 directly or via one way valve 214. In some embodiments, one way valve 214 is detachably attached to delivery port 202 or irremovably attached to port 202.

One way valve 214 includes access membrane 212 on its input side. Access membrane 212 comes in contact with means capable of delivering the medication 206 upon coupling. Access membrane 212 contains an antiseptic material that is capable of sterilizing means capable of delivering the medication 206 upon coupling.

One way valve 214 also includes means capable of delivering the medication 206, such as, a needle/catheter at the output side. The needle/catheter 206 can be detachably coupled to delivery port 200. One way valve 214 ensures unilateral flow into tubing 200 and prevents any back flow.

In one embodiment, as shown in FIG. 2B, medication container 106, one way valve 214, and tubing 200 are coupled and in liquid communication with each other. Medication container 106 is detachably attached to one way valve 214 and one way valve 214 is detachably attached to tubing 200.

In a second aspect the present invention is related to an intravenous tray. The tray can be adapted such that it can detachably couple to the chassis of the medication tray or the valve tray of the present invention. The intravenous tray includes a first input port for a fluid to come in and a second output port for the fluid to go out. The fluid can be a carrier fluid used for IV administration. The tray also includes a plurality of delivery ports arranged on the tray such that each delivery port is capable of fluidic contact with the first input port and the second output port. The delivery ports may be designed such that they are capable of detachably coupling to a plurality of means capable of delivering the medication. The delivery ports can also be designed such that they are fixed to the means capable of delivering the medication.

The intravenous tray can be slotted or coupled to the medication tray. It can be spiked into a carrier fluid, primed, and the tray slotted into the medication tray or the tray could be connected to a commercially available IV set. The intravenous tray can connect to the medication containers via a series of delivery ports. The delivery ports can contain one way valves preventing backflow of fluid into the containers, allowing for multiple uses of the containers for multiple patients without contamination. The intravenous tray can also incorporate the medication containers or the medication tray themselves.

In one embodiment, the intravenous tray has a docking capability for more medication trays or valve trays. The medication delivery system according to the present invention is modular in nature. It can be designed such that multiple modules or trays can be added to the system. In one embodiment an intravenous tray can be attached directly to the medication tray. In another embodiment an intravenous tray can be attached to the medication tray and also to a valve tray.

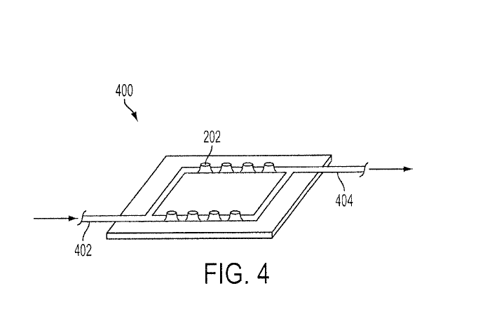

FIG. 4 shows an embodiment of an intravenous tray of the present invention. Intravenous tray 400 can be inserted into or coupled to a valve tray. Delivery ports 202 of the intravenous tray are capable of connecting to output port of a valve tray. A carrier fluid is capable of entering through input port 402 and coming out of output port 404.

The trays and the medication containers of the present invention can be disposable. After patient use, the trays, medication containers, and the IV tubing can be discarded. In one embodiment, the medication containers, since there has been no contamination secondary to the one way valves or valve trays, will remain in the system until they are empty and have to be replaced.

The different modules can be grouped into logical categories like: anesthesia, analgesia, resuscitative, epidural, ICU, general medical ward and the like.

The intravenous tray and the valve tray can be designed such that there is a means to sterilize the coupling components. The means to sterilize according to the present invention can be in the form of an access membrane or a material which can sterilize a port of the medication tray used for delivering medication. For example, the material can be selected such that it is capable of being infused with an antiseptic material.

The intravenous tray can include an access membrane which contains an antiseptic material. During coupling with a medication tray, which uses catheters as means for delivering the medication, the access membrane will rub on the catheter and sterilize the catheter by applying the antiseptic material on the catheter.

In a third aspect the present invention is related to a valve which is capable of coupling to the means capable of delivering the medication from the medication container and delivering the medication to the intravenous tubing or intravenous tray. The valve is capable of detachably coupling to said means or it can be fixed permanently to said means. The valve can be designed such that it allows unidirectional flow of medication from the medication container to the intravenous tubing or intravenous tray.

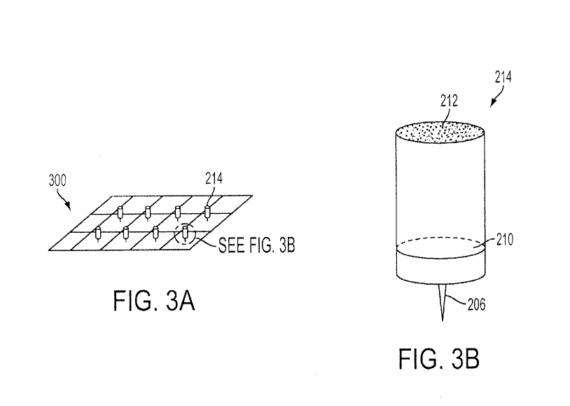

FIG. 3A shows a valve tray 300 which has eight one way valves 214. The valve tray could be coupled to a medication tray in order to connect the medication tray to the intravenous tray. Valve tray 300 could be made of plastic for structural integrity.

FIG. 3B shows a one way valve 214 that has an access membrane 212 on its input end, a one way valve 210 that ensures one way flow into the IV tubing and an output port 206. Access membrane 212 could be adapted or configured such that it contains an antiseptic material and sterilizes the means capable of delivering the medication upon

The valve can have a first input port which is capable of fluidic contact with the means capable of delivering the medication. In one embodiment the valve can include a first input port and a second output port such that the ports are capable of maintaining fluidic contact. The medication can enter from the first input port and go out of the second output port but not in the other direction. The valve can be designed such that it includes a means for sterilizing the means capable of delivering the medication. In one embodiment, the first input port of the detachable valve can be designed such that it contains a means to sterilize the medication container access port upon coupling.

In a fourth aspect the present invention is related to a valve tray which includes a plurality of detachable valves arranged on the valve tray. The valves are arranged on a valve tray such that the first input ports of a plurality of detachable valves are capable of coupling with a plurality of medication containers. The coupling can be achieved via container access ports of the medication containers. The medication container is capable of delivering the medication to the first input port of the valve tray which in turn can deliver the medication to an IV tubing.

The valve tray can be adapted such that it can detachably couple to the chassis of the medication tray. The valve tray can also be adapted such that it can detachably couple to the intravenous tray. In certain embodiments the valve tray can be designed such that it is fixed permanently to the intravenous tray. In one embodiment the output port of the valve tray is coupled to the delivery port of the intravenous tray. In certain other embodiments the valve tray can be designed such that it is fixed permanently to the medication tray such that the input port of the valve tray is coupled to the means for delivering the medication from the medication tray.

The valves can be detachably attached to the medication tray. The detachable valve can be attached to the means for delivering the medication via the input port of the valve. The output port of the valve can be attached to the intravenous tubing. The output port of the valve can also be designed such that it is capable of detachably coupling with the intravenous tubing. In one embodiment the output port of the valve can be attached to a delivery port of the intravenous tubing.

The valve can also be attached to the medication container such that it is capable of maintaining fluidic contact with the medication container. In one embodiment the valve is capable of maintaining fluidic contact with the medication container and the intravenous tubing. The valve can be designed such that it is capable of controllably allowing the medication to travel from the medication container into the intravenous tubing.

In one embodiment the medication tray can be adapted such that it includes an intravenous tubing in the form of an intravenous tray. The intravenous tray includes a first input port for a fluid to come in and a second output port for the fluid to go out. The tray also includes a plurality of delivery ports arranged on the tray such that each delivery port is capable of fluidic contact with the first input port and the second output port of the intravenous tray. The delivery ports may be designed such that they are capable of detachably coupling to a plurality of second output ports of valves of the present invention. The intravenous tray can also be designed such that it is permanently fixed to the valve tray.

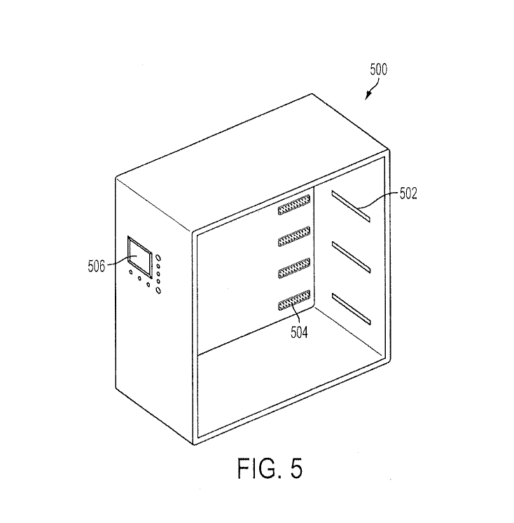

In a fifth aspect the present invention is related to a medication delivery unit which includes a housing adapted to hold at least one medication tray. The delivery unit can also include at least one module capable of coupling with the communication interface and the controller such that the controller is capable of transmitting and receiving information regarding a medication to be delivered and control delivery of the medication from the medication container to the intravenous tubing. The medication tray can be designed such that it can be ejected from the housing. FIG. 5 shows an embodiment of housing 500 adapted to hold medication delivery units of the present invention. Medication delivery unit housing 500 is designed to be modular with multiple slots 502 such that it is capable of accommodating multiple medication trays. Slot 502 is adapted to hold a medication tray. Medication delivery unit housing 500 includes multiple communication interfaces 504 that are capable of communicating with the communication interfaces of medication tray, an LCD panel 506, and control buttons. LCD panel 506 acts as a display interface or as a control unit. The LCD panel and control buttons can be adapted to provide a user access to and control over the medication delivery unit.

In one embodiment the medication delivery unit includes a capability to hold the medications for a prolonged amount of time. For example, the medication delivery unit can include a means to maintain the medication at a particular temperature. For example, a refrigeration unit can be coupled to the medication delivery unit.

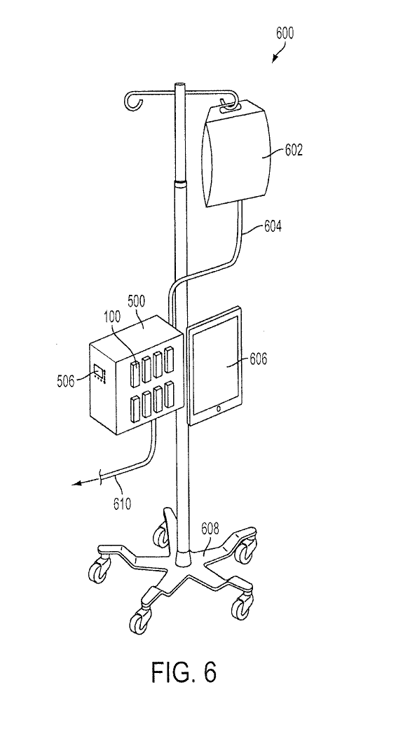

FIG. 6 shows one embodiment of a medication delivery unit 600 of the present invention. Medication delivery unit 600 includes an intravenous fluid containing bag 602. Bag 602 is connected to medication tray housing 500 via an IV tubing 604. Medication tray housing 500 has multiple medication trays 100 coupled to IV tubing 604 and in fluidic communication with bag 602. A controller 606 is connected to medication delivery unit 600.

Intravenous tubing 610 could be connected to a subject/patient in need of a medication. Medication delivery unit 600 could be assembled, for example, on a stand 608 in order to render it mobile for use, or it could be a standalone unit on rollers for greater mobility.

In a sixth aspect the present invention is related to a method of delivering a medication to a patient including loading the medication into the medication tray of the medication delivery unit of the present invention. The method also includes transmitting information regarding the medication to the controller.

The transmitted information can include information which identifies the medication to be delivered, patient identification, dosage of the medication or the location of the medication container containing the medication on the medication tray or the medication delivery unit. The step of transmitting information also includes transmitting information from a medical database to the controller and data from the controller, for example, a hospital pharmacy database or the electronic OR record keeper and the like.

The step of transmitting information also includes manually entering information at an interface connected to the controller. The information can be entered by an operator such as a medical professional. The interface connected to the controller can be a computer programmed to exchange information with the controller. The interface can be a part of the medication delivery unit.

The method includes delivering the medication from the medication delivery unit to the patient. The method also includes a verification step. The verification step can include, for example, verification of the identity of the medication, verification of the dosage of the medication, verification of the identity of the patient. The verification step can require an action by the operator which verifies the information or can require an action which verifies the information by comparing it with a medical database. In one embodiment the verification step includes checking the medication to be delivered against the patient's medical record which includes information such as allergies to medication, or prior delivered medication and the medications currently prescribed to the patient.

The delivery step according to the present invention includes delivering multiple dosages of the medication or delivering a continuous dose of the medication.

In a seventh aspect the present invention is related to a method of delivering a medication to a patient including loading the medication into the medication tray of the present invention. The method also includes transmitting information regarding the medication to the controller.

The controller according to the present invention is capable of controlling the identity of the medication delivered and/or the amount of medication delivered from the medication container to the intravenous tubing. The medication container can include an identifying mechanism such that the controller can identify the physical location of a medication on the medication tray. This can be achieved by, for example, having an individual electrical connector attached to each medication container which is in communication with the controller such that the controller can identify the medication and its physical location. The medication container can also be designed such that it includes a memory chip, which is accessible to the controller, which stores the identification information and the expiration date for the medication. The medication container and the included medication can be identified by, for example, a bar code, a QR code, a memory chip, a unique appendage attached to the container, a specific shape or color of the medication container. In one embodiment, the identification, for example, the code on the medication container or on the traditional vial can be scanned and the medication delivery unit would guide the operator to the slot or tray which accepts the medication. The medication unit can be designed such that it allows a particular slot to be occupied by a particular drug.

The controller according to the present invention is capable of communicating with the means capable of delivering the medication. The communication can be achieved via the communication interface of the medication tray or via a wireless link to the medication tray. The medication tray and/or the housing of the medication delivery unit of the present invention can include a transmitter/receiver which is capable of exchanging information with the controller. In one embodiment there can be a Bluetooth or wireless connectivity between the controller and the medication delivery unit allowing the control of the unit via, for example, a tablet computer or laptop from a remote location.

The controller according to the present invention can include an interface which can be operated by an operator such as a medical professional. The controller can be attached to the medication tray or to the medication delivery unit of the present invention. The interface can have a security feature which identifies the operator and authorizes use of the interface and/or connected medication delivery unit. The authorization can take place by comparing the security feature against, for example, an operator database which maintains information about individual operators. The operator database can be a central pharmacy database. The security features can be a pincode, an operator specific username and password, a fingerprint based authentication, a memory chip based authentication, a magnetic strip based authentication, a key based authentication and the like. Different operators can have different levels of authorizations, for example, a doctor can have a higher level of authorization than a support professional such as a nurse. The present invention also envisions a system where the controller can identify a group of operators and allow a particular group to deliver only certain medications. For example, an anesthesiologist is authorized to administer narcotics, but a nurse cannot. A group a operators such as nurses could have authorization to administer antibiotics. Moreover, the controller can be designed such that an operator profile is associated with each operator/user. The profile can be used to store and access operator/user information and preferences, such as, a layout of control screen, measurement units (metric/pounds, inches, degree Fahrenheit/degree Celsius etc.). The profile can also be used to store operator/user specific information such as preferred drugs used for standard general anesthetic or standard monitored anesthesia care (sedation), preferred concentrations of administered drugs, preferred rate of administration, and the like.

In one embodiment the controller is a computer which is capable of running proprietary software that creates a user interface using, for example, a touchscreen or laptop or menu of buttons with LCD readout which can be attached to the machine. It can be a computer which is programmed such that it is capable of receiving input from an operator.

An operator can enter patient specific data, for example, patient name, weight, height, age, allergies etc. into a computer running the software. Alternatively, the controller can receive all the appropriate patient data via the hospital intranet. The patient data, amongst other things, lets the controller calculate doses per weight, enter all the medication given into the patient chart and can also allow the controller to participate in automated OR record keeping. The controller can calculate the patient's body mass index (BMI) using patient specific data, such as weight and height. Moreover, the controller, based on the BMI, can analyze whether the patient is obese or not. For example, if BMI≧30, there could be a check box asking if patient is obese or not. If obese, the controller can further calculate lean body mass and percentage body fat of the patient using commonly available mathematical formulae. In obese patients, the amount of anesthetic, narcotic, and the like required can be calculated according to the patient's lean body mass. Also, the computer can have a lockout provision that prevents medication from being given to the patient which she/he is allergic to.

In one embodiment the controller is capable of receiving feedback input from at least one patient monitoring system such that the controller can modulate the delivery of the medication based on the feedback from the monitoring system. The patient monitoring system includes, for example, a BIS monitor, electrocardiogram, defibrillator, and oxygen monitor, end tidal CO₂ monitor, invasive and non-invasive blood pressure monitors, oxygen and volume sensors on ventilators, respiratory monitors and the like.

In one embodiment a train of four monitors, BIS monitors, SAO₂, blood pressure, ECG and defibrillators can be used to provide feedback. This enables the controller to give some drugs in response to signals received, for example, an increase in the train of four ratio can prompt the machine to administer an appropriate dose of nondepolarizing muscle relaxant if programmed to do so by the operator. Another example would be during a cardiopulmonary resuscitation, if the feedback shows that a shock has been given, there is no pulse and the ECG shows ventricular fibrillation the controller would administer the ads appropriate dose of epinephrine or vasopressin automatically.

Another example is in an ICU setting, where the drug delivery system can be interfaced with the invasive and noninvasive monitors hooked up to the patient. If the patient were receiving continuous intravenous pressor support, a drop in blood pressure would result in increasing the rate of infusion of the pressor, until the blood pressure had recovered to an acceptable value.

In one embodiment the controller can be attached to a medical database. The medical database can include, for example, information about various available medications, dosage information, compatibility information such as compatibility of one medication with another medication, expiration dates and life span of medication, delivery methods and the like. The medical database can also include the patient information, including medical records. For example, the controller exchanges information with intrahospital pharma software systems like PYXIS.

The controller will have access to a medication resource like the Physicians' Desk Reference (PDR), and is capable of providing relative dosing and medication information to the operator. It can also have the capability to use this data to warn the operator if, for example, the dose requested is outside the recommended dosing range. The unit can be designed such that an operator has to specifically confirm a desired dose. It will be possible to monitor and record all IV and parenteral medications administered hospital wide via the medication delivery systems. This would be of great value to for example hospital pharmacists or residency programs This will allow the hospital to keep up to the minute records of all the intravenous and intrathecal agents administered. It will improve supply logistics. It will also allow for better surveillance of controlled substances because the modules will be lockable, so that unauthorized persons cannot remove the drug containers.

In one embodiment, a doctor or a medical professional could order a medication, for example, a narcotic, on a hospital's intranet or via a remote connection, thereby unlocking the medication delivery unit for an authorized person, such as nurse, to administer the drug to a patient.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A medication tray comprising:
a chassis adapted to hold at least one medication container wherein the medication container comprises a medication and a means capable of delivering the medication from the medication container to intravenous tubing;
a communication interface capable of transmitting and receiving information and adapted to be in communication with the medication container and a controller wherein the controller is configured to process the information regarding the medication to be delivered and control delivery of the medication from the medication container to the intravenous tubing.

2. The medication tray according to claim 1, wherein the intravenous tubing has at least one delivery port capable of coupling with the means capable of delivering the medication.

3. The medication tray according to claim 1, wherein the intravenous tubing is in the form of an intravenous tray adapted to detachably couple with the chassis and comprising:
a first input port for a fluid to come in and a second output port for the fluid to go out;
a plurality of delivery ports arranged on the tray such that each delivery port is capable of fluidic contact with the first input port and the second output port; and
wherein a plurality of delivery ports are capable of detachably coupling to a plurality of means capable of delivering the medication.

4. The medication tray according to claim 3, wherein the delivery port further comprises a means to sterilize the means capable of delivering the medication.

5. The medication tray according to claim 1, wherein the means capable of delivering the medication is a container access port which allows flow of medication from the medication container to the intravenous tubing or a container valve which allows unidirectional flow of the medication from the medication container to the intravenous tubing.

6. The medication tray according to claim 5, wherein the container access port is adapted to couple with a first input port of a detachable valve wherein the valve allows unidirectional flow of the medication from the medication container to the intravenous tubing.

7. The medication tray according to claim 6, wherein the first input port of the detachable valve contains a means to sterilize the container access port upon coupling.

8. The medication tray according to claim 6, wherein a plurality of detachable valves are arranged on a valve tray such that the first input ports of a plurality of detachable valves are capable of coupling with container access ports of a plurality of medication containers.

9. The medication tray according to claim 8, wherein the valve tray is adapted to detachably couple to the chassis.

10. The medication tray according to claim 8, wherein each detachable valve comprises a second output port which is capable of detachably coupling with the intravenous tubing.

11. The medication tray according to claim 10, wherein the intravenous tubing is in the form of an intravenous tray adapted to detachably couple with the valve tray and comprising:
a first input port for a fluid to come in and a second output port for the fluid to go out;
a plurality of delivery ports arranged on the tray such that each delivery port is capable of fluidic contact with the first input port and the second output port; and
wherein a plurality of delivery ports are capable of detachably coupling to a plurality of second output ports of the detachable valves.

12. The medication tray according to claim 1, wherein the controller is capable of controlling the identity of the medication delivered and/or the amount of medication delivered from the medication container to the intravenous tubing.

13. The medication tray according to claim 1, wherein the controller is in communication with the means capable of delivering the medication via the communication interface.

14. The medication tray according to claim 1, wherein the controller is in capable of receiving input from a device capable of transmitting information regarding the medication to be delivered.

15. The medication tray according to claim 1, wherein the controller is capable of receiving input from an operator.

16. The medication tray according to claim 1, wherein the medication container can be ejected from the chassis.

17. A medication delivery unit comprising:
a housing adapted to hold at least one medication tray according to claim 1; and
at least one module capable of coupling with the communication interface and the controller such that the controller is capable of transmitting and receiving information regarding a medication to be delivered and control delivery of the medication from the medication container to the intravenous tubing.

18. The medication delivery unit according to claim 17, wherein the medication tray can be ejected from the housing.

19. The medication delivery unit according to claim 17, wherein the controller is capable of receiving feedback input from at least one physiological monitoring system such that the controller can modulate the delivery of the medication based on the feedback from the monitoring system.

20. The medication delivery unit according to claim 19, wherein the physiological monitoring system is selected from a group consisting of BIS monitor, electrocardiogram, defibrillator, oxygen monitor, end tidal CO₂ monitor, invasive and non-invasive blood pressure monitors, oxygen sensors, volume sensors, and respiratory monitors.

21. A method of delivering a medication to a patient comprising:
loading the medication into the medication tray of the medication delivery unit of claim 17; and
transmitting information regarding the medication to the controller.

22. The method according to claim 21, further comprising delivering said medication from the medication delivery unit to the patient.

23. The method according to claim 21, wherein said transmitted information identifies the medication to be delivered, patient identification, dosage of the medication or the location of the medication container containing the medication.

24. The method according to claim 21, wherein the step of transmitting information comprises transmitting information from a medical database to the controller.

25. The method according to claim 21, wherein the step of transmitting information comprises manually entering information at an interface connected to the controller.

26. The method according to claim 25, wherein the interface connected to the controller can be a computer programmed to exchange information with the controller.

27. The method according to claim 22, wherein the step of delivering comprises delivering multiple dosages of the medication or delivering a continuous dose of the medication.

28. The method according to claim 21, further comprising a verification step requiring operator intervention.

29. The method according to claim 28, wherein the verification step comprises verification of the identity of the medication.

30. The method according to claim 28, wherein the verification step comprises verification of the dosage of the medication.

31. The method according to claim 28, wherein the verification step comprises verification of the identity of the patient.

32. A method of delivering a medication to a patient comprising:
loading the medication into the medication tray of claim 1; and
transmitting information regarding the medication to the controller.
